(19) United States
(12) Patent Application Publication
(10) Pub. No.: US 2025/0018075 A1
(43) Pub. Date: Jan. 16, 2025

(54) AIR-DRIVEN PLASMACLUSTER SUPEROXIDE CLOUD

(71) Applicant: PCS Biosecurity, Corp, Kansas City, MO (US)

(72) Inventor: David R. Ogle, Lawrence, KS (US)

(73) Assignee: PCS Biosecurity, Corp, Kansas City, MO (US)

(21) Appl. No.: 18/714,515

(22) PCT Filed: Nov. 28, 2022
(86) PCT No.: PCT/US22/51092

§ 371 Date: May 29, 2024

Related U.S. Application Data
(60) Provisional application No. 63/284,458, filed on Nov. 30, 2021.

(51) Int. Cl. A61L 2/14; A61L 2/00; A61L 2/26; F24F 8/26

(52) U.S. Cl. CPC A61L 2/14; A61L 2/0011; A61L 2/26; F24F 8/26; A61L 2202/11; A61L 2202/15

(57) ABSTRACT

A process and system comprising a plasmacluster cassette fan; a negative air anion device, said negative air anion device emit negative ions into the air/surroundings; an accessory filter bank, said accessory filter bank is configured to filter room or supply air directed to said plasmacluster cassette fan and negative air anion device; an evaporator heat exchanger; and a supplementary fan, said supplementary fan is configured to distribute the Plasmacluster Superoxide Cloud or negative air anions to targeted specific medias.

### CROSS-REFERENCE TO RELATED APPLICATIONS

The present Utility patent application is the National Phase filing under 35 U.S.C. 371 of the International Application No PCT/US22/51092 filed Nov. 28, 2022, entitled “Air-Driven Plasmacluster Superoxide Germicidal Cloud”, which claims priority benefit of the U.S. provisional patent application No. 63/284,458, entitled “Air-Driven Plasmacluster Superoxide Germicidal Cloud” filed on 30 Nov. 2021 under 35 USC 119(e). The contents of these related applications are incorporated herein by reference for all purposes to the extent that such subject matter is not inconsistent herewith or limiting hereof.

### BACKGROUND

One or more embodiments of the invention generally relate to a process involving moving a volume of air. More particularly, certain embodiments of the invention relate to a system and process involving moving specific volume of air with a myriad of fan and blower technologies with a given CFM (cubic feet per minute), at a very specific flow rate FPM (feet per minute or velocity) to achieve a Plasmacluster of Negative Air-Anions for the targeted concentration of Negative Air-Anions per cubic centimeter (cc) of air in the Plasmacluster Cloud made by a Negative Air-Anion device directly deployed and distributed towards the subject or media.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that anions, negatively charged electrons, generated by plasmaclustering devices have lower density than standard atmospheric air (1.225 kg/m\hat{ }3)1. With lower density, negative air-anions are subject to buoyancy, an upward force that causes them to rise in the air. This same phenomenon explains why lighter objects would rise to the surface in a body of water if placed at the very bottom, and continue to float at the surface (i.e., life jackets, buoys, and other flotation devices). Using negative air-anion sensors, the rate of buoyancy was found to be approximately 2 feet per second thus modeled by the equation h(t)=2t.

Negative air-anions are lighter than the surrounding air, resulting in buoyancy requiring additional calculations for direct deployment. Secondly, plasmaclusters are affected by decay, a factor of time. Thirdly, plasmaclusters are affected by attrition, a measure of friction. Fourthly, air volume and air velocity greatly affect the Plasmacluster concentration and distance the cluster can travel. Finally, different fan technologies affect the Plasmacluster in concentration and distance the Plasmacluster Superoxide Cloud may travel based on air capacity and velocity.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is an illustration of an exemplary air-driven plasmacluster superoxide cloud, in accordance with an embodiment of the present invention;

FIG. 2 is an illustration of an exemplary direct deployment of plasmacluster superoxide cloud in HVAC system, in accordance with an embodiment of the present invention; and

FIG. 3 is an illustration of an exemplary direct deployment of plasmacluster superoxide cloud in refrigeration systems, in accordance with an embodiment of the present invention.

Unless otherwise indicated illustrations in the figures are not necessarily drawn to scale.

### DETAILED DESCRIPTION

The present invention is best understood by reference to the detailed figures and description set forth herein.

Plasmacluster ions are the same type of positive and negative ions that occur in nature. After purifying the air, the ions turn to water and return to the air.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial,” “nearly,” “almost,” “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user,” or any similar term, as used herein, may mean a human or non-human user thereof. Moreover, “user,” or any similar term, as used herein, unless expressly stipulated otherwise, is contemplated to mean users at any stage of the usage process, to include, without limitation, direct user(s), intermediate user(s), indirect user(s), and end user(s). The meaning of “user,” or any similar term, as used herein, should not be otherwise inferred or induced by any pattern(s) of description, embodiments, examples, or referenced prior-art that may (or may not) be provided in the present patent.

References to “end user,” or any similar term, as used herein, is generally intended to mean late-stage user(s) as opposed to early-stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising” And “contain” and variations of them—Such terms are open-ended and mean “including but not limited to.” When employed in the appended claims, this term does not foreclose additional structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn. 112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus, in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or subcombination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components is described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

In some embodiments of the present invention and variations thereof, relate to a system and process involving moving specific volume of air with a myriad of fan and blower technologies. In one embodiment of the present invention the system and process involving moving specific volume of air with a myriad of fan and blower technologies with a given CFM (cubic feet per minute), at a very specific flow rate FPM (feet per minute or velocity) to achieve a Plasmacluster of Negative Air-Anions for the targeted concentration of Negative Air-Anions per cubic centimeter (cc) of air in the Plasmacluster Cloud made by a Negative Air-Anion device directly deployed and distributed towards the subject or media. Plasmaclusters density calculated by airflow and air volume of at least 10,000 negative air-anions/cc are considered germicidal, however a concentration of at least 50,000 negative air-anions/cc in the directly deployed air is required to be a superoxide.

In some embodiment, the Plasmacluster Superoxide Negative Air-Anion Cloud is targeted at specific medias including (but not limited to) people, livestock, viruses, bacteria, mold, mildew, heat exchangers in HVAC and refrigeration systems, or an agar dish for testing resulting in a Superoxide germicidal disinfectant enveloping the subject(s) and or media(s).

In other embodiments, direct deployment of a Plasmacluster Superoxide Cloud towards the subject is the most effective method of its germicidal capability. Any sort of friction from obstructions such as conduit, pipes, objects, walls, or ducting causes excessive attrition and decays the Plasmacluster potential, lowering the number of Negative Air-Anions/cc decreasing the concentration. Examples of direct deployment could be showering the Plasmacluster Superoxide on to a patient and doctor in a hospital room, directly deploying above a door in an office building blowing down, from the side laterally into a crowd of people indoors, directly at any subject matter or object or using an auxiliary fan in HVAC systems. Direct deployment of the Plasmacluster Superoxide Cloud uses limited conduit or ducting to avoid friction losses (attrition) and Negative Air-Anion devices are always placed on the discharge side of the fan or blower.

In the use regarding HVAC systems, direct deployment using another embodiment would be directed at the evaporator heat exchanger using an auxiliary Plasmacluster cassette fan, adding a second fan beyond the service fan of the HVAC ducting in the return air of the HVAC system. Because of the nature of modern HVAC systems, ducting is provided by the system itself and acts as a conduit to direct the Plasmacluster Superoxide Cloud. The added filter bank filters the room air directed to the Plasmacluster Cassette with the Negative Air Anion device. In the use regarding refrigeration case cooler direct deployment, an auxiliary fan Plasmacluster Cassette fan is used beyond the service fan of the refrigerator evaporator coil.

In some embodiments, the unique and calculated airflow/air volume process for the precise direct deployment concentration of the Plasmacluster Negative Air-Anion Superoxide Cloud enveloping the herein listed media and subjects.

The present invention will now be described in detail with reference to embodiments thereof as illustrated in the accompanying drawings.

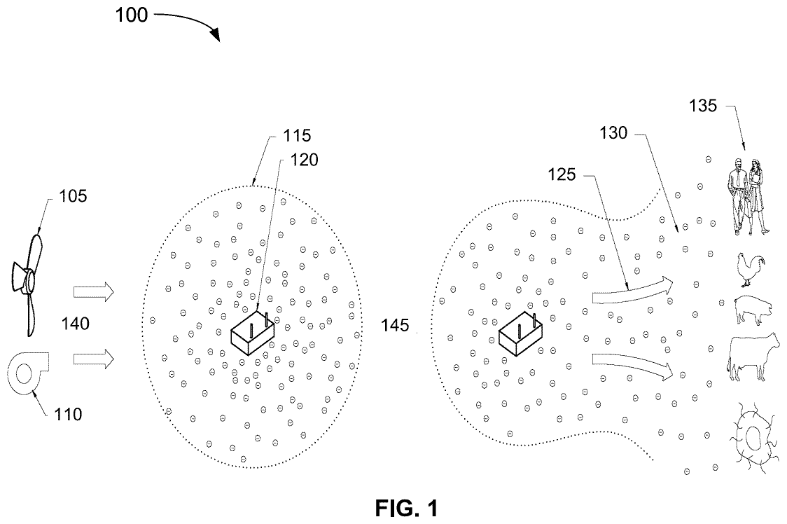

FIG. 1 is an illustration 100 of an exemplary air-driven plasmacluster superoxide cloud, in accordance with an embodiment of the present invention. In one embodiment of the present invention, the process of deploying plasmacluster superoxide cloud is by creating airflow 140 with an airflow source 105 110 in a calculated manner to a negative anion field 115 generated by a plasmacluster device 120. Superoxide cloud greater than 50,000 negative air anions/cc 130 may be directly deployed and distributed to people 135, working environments, livestock, viruses, bacteria, mold, mildew, agar dishes for testing, or other media resulting in an enveloping superoxide disinfectant as a result of direct deployment 145.

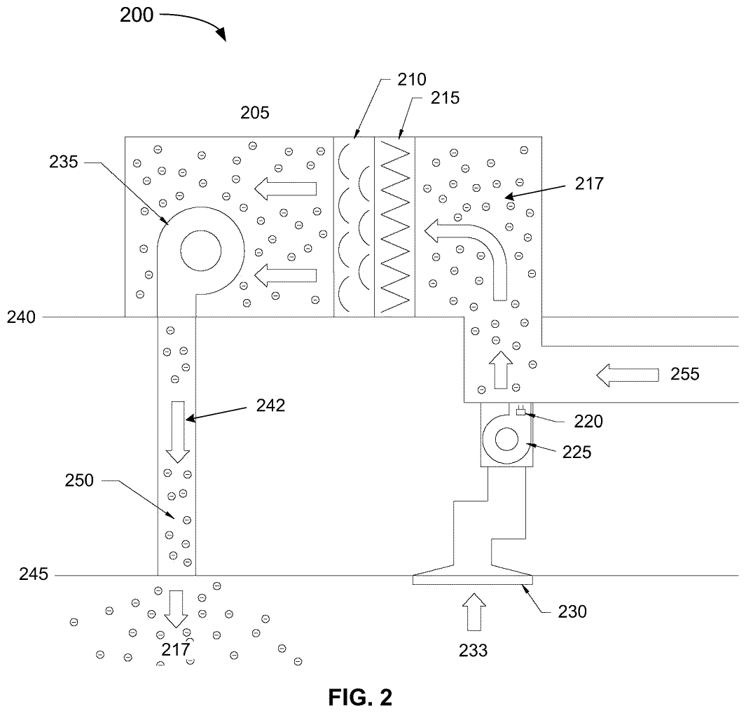

FIG. 2 is an illustration of an exemplary direct deployment of plasmacluster superoxide cloud in an HVAC system 200, in accordance with an embodiment of the present invention. In one embodiment of the present invention, direct deployment may be directed at an evaporator heat exchanger 210 using an auxiliary Plasmacluster cassette fan 225 and adding a supplementary fan 235 beyond the service fan of the HVAC ducting 253 in the return air 255 of the HVAC system. Because of the nature of modem HVAC systems, ducting is provided by the system itself and acts as a conduit to direct Plasmacluster Superoxide Cloud 217 through a rooftop unit 205 and into an outlet supply air vent 250, as indicated by arrows 242. In other embodiments, HVAC system 200 may include but not limited to an HVAC rooftop unit containing evaporator heat exchanger 210, subsidiary filter bank 215, and supplementary fan 235. Supplementary fan 235 may include but not limited to Plasmacluster cassette fan. In use room or supply air 233 enters an air inlet vent 227 with an accessory filter bank 230. Accessory filter bank 230 filters room or supply air 233 directed to Plasmacluster Cassette fan 225 and Negative Air Anion device 220. Negative Air Anion device 220 may emit negative ions into the air/surroundings. Plasmacluster Superoxide Cloud or negative air anions 217 generated by Negative Air Anion device 220 may be directed to a second filter bank 215 and evaporator heat exchanger 210. A Plasmacluster Superoxide Cloud or negative air anions density may be based on airflow and air volume taking into account attrition and decay factors of the plasmaclusters. Supplementary fan 235 may push or distribute the Plasmacluster Superoxide Cloud or negative air anions 217 to outlet supply air vent 250 to be distributed to targeted specific medias. HVAC system 200 may comprise but not limited to split systems, hybrid systems, duct-free systems, and/or packaged heating and air systems incorporating Plasmacluster Cassette fan 225, Negative Air Anion device 220, and second fan 235.

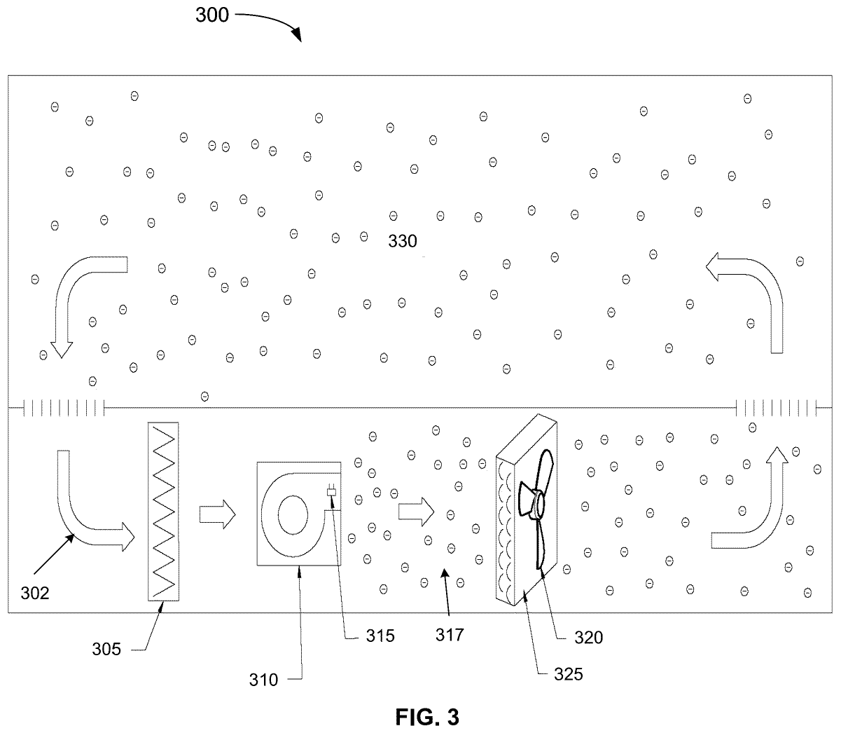

FIG. 3 is an illustration of an exemplary direct deployment of plasmacluster superoxide cloud in refrigeration systems 300, in accordance with an embodiment of the present invention. In one embodiment of the present invention, supply air 302 may be supplied to an inlet filter 305 to be directed to Plasmacluster Cassette fan 310 and Negative Air Anion device 315. Plasmacluster Superoxide Cloud or negative air anions 317 generated by Negative Air Anion device 315 may be directed to a refrigerator service or supplementary fan 320 and evaporator cooler or evaporator heat exchanger 325 before supplying the Plasmacluster Superoxide Cloud or negative air anions to a refrigeration case or compartment cooler 330, via a cooled air conveyor or entry vent 335, that may contain fruits, vegetables, perishables, etc. Refrigerator service fan 320 and evaporator cooler 325 may be parts of an existing refrigeration unit where Plasmacluster Cassette fan 310 and Negative Air Anion device 315 are incorporated. Refrigeration systems 300 may comprise but not limited to Mechanical-Compression Refrigeration Systems, Absorption Refrigeration, Evaporative Cooling, Refrigeration case cooler and/or Thermoelectric Refrigeration incorporating Plasmacluster Cassette fan 310 and Negative Air Anion device 315. Supply or cool air 302 enters and exits the refrigeration compartment 330 from the cooling or freezer compartment 327 through entry vent 335 and exit vent 340.

All the features disclosed in this specification, including any accompanying abstract and drawings, may be replaced by alternative features serving the same, equivalent, or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of implementing Air-Driven Plasmacluster Superoxide Germicidal Cloud according to the present invention will be apparent to those skilled in the art. Various aspects of the invention have been described above by way of illustration, and the specific embodiments disclosed are not intended to limit the invention to the particular forms disclosed. The particular implementation of the Air-Driven Plasmacluster Superoxide Germicidal Cloud may vary depending upon the particular context or application. By way of example, and not limitation, the Air-Driven Plasmacluster Superoxide Germicidal Cloud described in the foregoing were principally directed to systems and processes involving moving specific volume of air with a myriad of fan and blower technologies with a given CFM (cubic feet per minute), at a very specific flow rate FPM (feet per minute or velocity) to achieve a Plasmacluster of Negative Air-Anions for the targeted concentration of Negative Air-Anions per cubic centimeter (cc) of air in the Plasmacluster Cloud made by a Negative Air-Anion device directly deployed and distributed towards the subject or media implementations; however, similar techniques may instead be applied to disinfecting air supply, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

Only those claims which employ the words “means for” or “steps for” are to be interpreted under 35 USC 112, sixth paragraph (pre-AIA) or 35 USC 112(f) post-AIA. Otherwise, no limitations from the specification are to be read into any claims, unless those limitations are expressly included in the claims.

What is claimed is:

1. A system comprising:
a plasmacluster cassette fan;
a negative air anion device, said negative air anion device emit negative ions into the air/surroundings;
an accessory filter bank, said accessory filter bank is configured to filter room or supply air directed to said plasmacluster cassette fan and negative air anion device;
an evaporator heat exchanger; and
a supplementary fan, said supplementary fan is configured to distribute the Plasmacluster Superoxide Cloud or negative air anions to targeted specific medias.

2. The system of claim 1, further comprising a subsidiary filter bank.

3. The system of claim 1, further comprising an inlet air supply vent for receiving the room or supply air.

4. The system of claim 1, further comprising return air ducting.

5. The system of claim 1, further comprising a rooftop unit.

6. The system of claim 1, further comprising an outlet supply air vent configured to distribute the Plasmacluster Superoxide Cloud or negative air anions to targeted specific medias.

7. The system of claim 1, further comprising a refrigerator service fan.

8. The system of claim 7, further comprising an evaporator cooler or evaporator heat exchanger.

9. The system of claim 8, further comprising a cooled air conveyor or entry vent.

10. The system of claim 9, further comprising a refrigeration case cooler.

11. The system of claim 10, further comprising an exit vent.

12. The system of claim 11, further comprising a freezer compartment.

13. A system comprising:
a plasmacluster cassette fan;
a negative air anion device, said negative air anion device emit negative ions into the air/surroundings;
an accessory filter bank, said accessory filter bank is configured to filter room or supply air directed to said plasmacluster cassette fan and negative air anion device;
an evaporator heat exchanger;
a subsidiary filter bank;
a supplementary fan, said supplementary fan is configured to distribute the Plasmacluster Superoxide Cloud or negative air anions; and
an outlet supply air vent configured to distribute the Plasmacluster Superoxide Cloud or negative air anions to targeted specific medias.

14. The system of claim 13, further comprising an inlet air supply vent for receiving the room or supply air.

15. The system of claim 14, further comprising return air ducting.

16. The system of claim 15, further comprising a rooftop unit.

17. A system comprising:
a plasmacluster cassette fan;
a negative air anion device, said negative air anion device emit negative ions into the air/surroundings;
an accessory filter bank, said accessory filter bank is configured to filter room or supply air directed to said plasmacluster cassette fan and negative air anion device;
an evaporator heat exchanger;
a supplementary fan, said supplementary fan is configured to distribute the Plasmacluster Superoxide Cloud or negative air anions;
a refrigeration case cooler; and
a freezer compartment.

18. The system of claim 17, further comprising a cooled air conveyor or entry vent.

19. The system of claim 18, further comprising an exit vent.

20. The system of claim 19, further comprising a refrigeration case cooler.
